(19) United States
(12) Patent Application Publication
(10) Pub. No.: US 2015/0253301 A1
(43) Pub. Date: Sep. 10, 2015

(54) IN VITRO METHOD AND APPARATUS FOR DETERMINING EFFICACY AND ACTION MECHANISMS OF A TOPICAL COMPOSITION ON VARIOUS SKIN COLOR TYPES

(71) Applicant: AKZO NOBEL SURFACE CHEMISTRY LLC, Chicago, IL (US)

(72) Inventors: Olga V. DUEVA-KOGANOV, White Plains, NY (US); Michael KOGANOV, White Plaiins, NY (US); Artyom DUEV, White Plaiins, NY (US)

(73) Assignee: AKZO NOBEL SURFACE CHEMISTRY LLC, Chicago, IL (US)

(21) Appl. No.: 14/719,915

(22) Filed: May 22, 2015

Related U.S. Application Data
(63) Continuation of application No. 13/151,787, filed on Jun. 2, 2011.
(60) Provisional application No. 61/350,577, filed on Jun. 2, 2010.

(51) Int. Cl. G01N 33/483; G01N 21/29

(52) U.S. Cl. CPC G01N 33/4833; G01N 21/293; G01N 2500/20; G01N 2500/10; G01N 2201/061

(57) ABSTRACT

The present invention relates to in vitro methods for determining efficacy of topical compositions on various skin color types. These methods involve providing an artificial skin apparatus configured to approximate the color and diffuse reflectance characteristics of a predetermined human skin color type. The artificial skin apparatus includes an artificial skin substrate combined with a color background, with the color background correlating to the human skin color type. A topical composition of interest is applied to the artificial skin substrate of the artificial skin apparatus and then pre-irradiated. The pre-irradiated topical composition is then analyzed using relevant experimental techniques or assays for at least one efficacy parameter, including, for example, anti-ageing, photoprotective, sun protective, UVA/UVB protective, UVAI protective, photo stabilizing, and photosensitizing activities and action mechanisms of the topical composition on various skin color types. Also provided is an artificial skin apparatus and kit for use with the in vitro method.

### CROSS-REFERENCE TO RELATED APPLICATIONS

This application claims priority benefit of U.S. Provisional Patent Application Ser. No. 61/350,577, filed Jun. 2, 2010, the disclosure of which is hereby incorporated by reference herein in its entirety.

### FIELD OF THE INVENTION

The present invention relates to an in vitro method and apparatus for determining efficacy and mechanisms of action of a topical composition on various skin color types.

### BACKGROUND OF THE INVENTION

It is known that exposure to sunlight can result in a wide range of adverse health consequences. Sun light that reaches the earth has different amounts of UVC, UVB, UVA, visible, and infrared radiation. 100% of UVC (λ<290 nm), 95% of UVB (290<λ<320 nm) and only 5% of UVAII and UVAI (320<λ<400) are filtered by the atmosphere. Excessive exposure to UVB light (290-320 nm) can have both short and long-term effects. The immediate and primary consequence of unprotected UVB exposure is erythema and sunburn. Examples of longer term consequences, childhood sunburns, have been correlated with melanoma later in life. UVA light (320-400 nm) penetrates deeper than UVB, reaching both the epidermis and dermis. Repeated exposure to the shorter wavelength UVA II rays (less than about 320-340 nm) and the longer wavelength UVA I rays (340-400 nm) have been associated with extrinsic skin ageing manifested in formation of fine lines and wrinkles, irregular skin pigmentation, and weakening of the skin's immune system. Other skin disorders associated with sunlight include basal cell carcinomas and squamous cell carcinomas, actinic keratoses and premature aging of the skin.

Photodamage to the skin can be caused by full spectrum solar radiation in the range of 290-800 nm and involves free-radical mechanism; free radicals are generated near the skin surface by the interaction of radiation with the substrate and diffuse into subsurface region to cause damage [Yash K. Kamath and Sigrid B. Ruetsch. Characterization of surface and subsurface photodegradation of skin. IFSCC Magazine—vol. 6, no 3/2003: 200-204].

Sun exposure leads to generation of reactive oxygen species (ROS) in the skin. ROS are cytotoxic and can be classified into radical (e.g. superoxide and hydroxyl radical) and non-radical (e.g. hydrogen peroxide, singlet oxygen and peroxynitrile) species [Gracy R W, Talent J M, Kong Y, Conrad C C. Reactive oxygen species: the unavoidable environmental insult. Mutat Res. 1999 Jul. 16; 428 (1-2): 17-22; and Fujita T, Fujimoto Y. Formation and removal of active oxygen species and lipid peroxides in biological systems. Nippon Yakurigaku Zasshi. 1992 June; 99(6): 381-9].

The process of ROS production is different in each wavelength region. UVB is efficiently absorbed by biomolecules that are present in skin, inducing photochemical reactions that result in direct damage to them. In the case of UVA and visible light, few molecules actually absorb this radiation (derivatives of flavins, e.g. riboflavin, porphyrins, and melanin) and the production of ROS and reactive nitrogen species (RNS) is accomplished mostly by photosensitization [F. Wilkinson, W. P. Helman, and A. B. Ross. Quantum yields for the photosensitized formation of the lowest electronically excited singlet state of molecular oxygen in solution. J. Phys. Chem. Ref Data, 22, 113-262 (1993)].

It was demonstrated that 60 minutes after application sunscreen filters octocrylene, octinoxate and oxybenzone can enhance UV-induced ROS generation determined by fluorescence in epidermal skin model [Kerry M. Hanson, Enrico Gratton, Christopher J. Bardeen. Sunscreen enhancement of UV-induced reactive oxygen species in the skin. Free Radic Biol Med. 2006 (41): 1205-1212].

Free radical formation occurs in epidermis and dermis at all UV and VIS wavelengths over all sun spectrum; sunscreen products should be designed with antioxidants or radical scavengers in order to ensure sufficient radical protection [Leonard Zastrow et al. Detection and identification of free radicals generated by UV and visible light in ex vivo human skin. IFSCC Magazine—vol. 11, no 3/2008: 207-215].

The direct or indirect attack of ROS on essential constituents of biological membranes has been shown to result in the formation of a number of peroxidative lipid breakdown-products: lipid hydroperoxide, lipid peroxyl radical and lipid alkoxyl radical [Fujita T, Fujimoto Y. Formation and removal of active oxygen species and lipid peroxides in biological systems. Nippon Yakurigaku Zasshi. 1992 June; 99(6): 381-9].

UVA produced a dose-dependent linear increase of lipid peroxidation in liposomal membrane, as detected by the assay of malondialdehyde [Biplab Bose, Sanjiv Agarwal and S. N. Chatterjee. UV-A induced lipid peroxidation in liposomal membrane. Radiat Environ Biophys (1989) 28: 59-65].

UVA radiation-generated singlet oxygen reacts with phosphatidylcholine to form lipid hydroperoxides; both are important redox active species involved in the deleterious effects of UVA radiation on lipids [Glenn F. Vile and Rex M. Tyrrell. Uva radiation-induced oxidative damage to lipids and proteins in vitro and in human skin fibroblasts is dependent on iron and singlet oxygen. Free Radical Biology and Medicine Volume 18, Issue 4, April 1995: 721-730].

UVB and UVC irradiation of phospholipid liposomes in conjunction with TBAR and TLC assays was utilized to assess the effects of antioxidants on lipid peroxidation in exposed (irradiated) liposomes [Edward Pelle et al. An in vitro model to test relative antioxidant potential: ultraviolet-induced lipid peroxidation in liposomes. Archives of Biochemistry and Biophysics Vol. 283, No. 2, December 1990: 234-240].

Singlet oxygen is responsible for much of the physiological damage caused by ROS and its lifetime is sufficiently long to permit significant diffusion in cells and tissues [Rodgers M A J, Snowden P T. Lifetime of O2(1δg) in Liquid Water as Determined by Time-Resolved Infrared Luminescence Measurements. J Am Chem Soc (1982) 104:5541-5541].

Singlet oxygen is linked with the in vivo UVA action spectrum, which is responsible for photoaging of skin [Kerry M. Hanson and John D. Simon. Epidermal trans-urocanic acid and the UV-A-induced photoaging of the skin. PNAS Sep. 1, 1998, vol. 95, No. 18:10576-10578].

Taken together with the present observation that UVA radiation-induced singlet oxygen is capable of generating mitochondrial DNA mutations in UVA-irradiated dermal fibroblasts, it is possible that the generation of singlet oxygen in human skin is of central importance for photoaging. Singlet oxygen quenching may thus represent an effective strategy to protect human skin from photoaging [Mark Berneburg et al. Singlet Oxygen Mediates the UVA-induced Generation of the Photoaging-associated Mitochondrial Common Deletion. May 28, 1999 The Journal of Biological Chemistry, 274: 15345-15349].

The amount of light necessary to maintain normal functionality of the dermis without harming the skin is basically unknown and is certainly dependent on the skin characteristics inherent to each individual. Therefore, besides protecting the skin from light by using sun-blocking agents, it is important to consider other strategies including processes that aim to facilitate maintenance of the redox balance [Mauricio da Silva Baptista. Photochemistry, Photobiology, and Redox Balance in Skin and Hair. Part I. www.nyscc.org/cosmetiscope/backissues/Cosmetiscope_{—}01.2011_FINAL.pdf].

Better understanding of the photobiological effects that UV radiation exerts on human skin and whether antioxidants could affect UV-filter photosensitized ROS generation will help to improve the quality of sunscreen products and foster the development of antioxidants and active agents that can be used in combination with sunscreen filters to provide better photoprotection for human skin [Kerry M. Hanson, Enrico Gratton e al. Sunscreen enhancement of UV-induced reactive oxygen species in the skin. Free Radic Biol Med. 2006 (41): 1205-1212; and Jean Krutmann. New Developments in Photoprotection of Human Skin. Skin Pharmacology and Applied Skin Physiology 2001; 14: 401-407].

Sunscreens are used to protect the human skin against harmful UVA/UVB radiation. Currently there is a trend toward higher sun protection factors (SPF) against UVB radiation and sufficient UVA protection. In vitro and in vivo methods for the assessment of efficacy and photostability of sunscreen products involve irradiation step, and the photostability properties of the sunscreen formulation have an influence on its overall efficacy in vivo and in vitro. For example, a pre-irradiation is used as an essential step of sunscreen's UVA in vitro testing methodologies. The COLIPA (European Cosmetics Trade Association) in vitro method for measuring UVA protection is used in European geographies for testing and labeling UVA efficacy of sunscreen products after pre-irradiation [P. J. Matts et. al. The COLIPA in vitro UVA method: a standard and reproducible measure of sunscreen UVA protection. Int. Journal of Cosmetic Science 2010, 32, 35-46]. The FDA's (US Food and Drug Administration) Proposed Rules on UVA protection offer a comprehensive evaluation of sunscreen product efficacy in vivo (SPF and UVA-PF) and in vitro (UVAI/UV ratio) after pre-irradiation [FDA 21 CFR Parts 347 and 352. Proposed Rules, Federal Register, §352.1, 72(165), 49070-49122 (2007)]. The Boots UK limited star rating system (2008 revision), a proprietary in vitro method used in the UK and Ireland to describe the ratio of UVA to UVB after pre-irradiation step. Comparison of these UVA in vitro test methods for the assessment of efficacy and photostability of sunscreen products is presented in Table I.

TABLE I
In vitro UVA Methods for the Assessment of Efficacy and Photostability of
Sunscreen Products

| | FDA Proposed Rule | | Boots star rating system |
|---|---|---|---|
| Parameters | (Aug. 27, 2007) | COLIPA (2009) | (2008 Revision) |
| Pre-Irradiation | Equal to SPF of sunscreen | D (dose) = UVAPF0 × D0 | 17.5 J/cm² (representing 60 minutes |
| Dose | product multiplied by 200 J/m²- | J/cm²; D0 value is fixed at 1.2 J/cm² | of ‘standard’ sun equivalent UVA). |
| | eff multiplied by 2/3. | UVA. | |
| Irradiation Source | Pre-irradiation dose (PID) | As similar as possible to the | The exposure source should be as |
| | in terms of “erythemal | irradiance at ground level | similar as possible to COLIPA (1994) |
| | effective dose” in order to | under a standard zenith sun as | reference spectrum |
| | allow various solar | defined by COLIPA (1994) or | |
| | simulators to be used. | in DIN 67501 (1999). | |
| Irradiation Flux | Not Specified | Total UV irradiance (290 to | Total UV irradiance (290 nm to |
| | | 400 nm) 50-140 W/m2; | 400 nm) should not be less than |
| | | Irradiance ratio of UVA (320 | 45 W/m2 and should not exceed |
| | | to 400 nm) to UVB (290 to | 75 W/m2. The UVA (320 nm to |
| | | 320 nm) 8-22. | 400 nm) irradiance must be no less |
| | | | than 90% and no more than 97% of |
| | | | total UV irradiance. |
| Sunscreen | 2 mg/sq. cm | 0.75 mg/sq. cm | 1 mg/sq. cm |
| Application Dose | | | |
| UVA Efficacy | UVAI/UV Ratio | (a) Ratio of UVA PF to SPF | UVA/UVB Ratio |
| parameter | | (b) Critical Wavelength | |
| Criteria | >0.2 to >0.95 | (a) at least 1/3; (b) >370 nm | 0.61 to >0.91 |
| Label Claim | 1 to 4 stars | UVA logo | 3 to 5 stars |
| Substrate | Quartz. The FDA requested | PMMA | Quartz or PMMA. Alternatives may |
| | comment regarding | | be used as substrate if their suitability |
| | suitability of other possible | | was demonstrated. |
| | substrates | | |
| Temperature of | Not specified | Less than 40 deg. C. | Within a range from 20 deg. C. to 40 |
| the substrate | | | deg. C. |
| Background on | Not specified | Dark (black) background | Not specified |
| which substrate | | | |
| is placed | | | |

In these UVA in vitro test methods presented in Table I, a background on which substrate is placed during pre-irradiation step is either black or dark (COLIPA 2009) or not specified at all.

In other existing photostability testing methodologies in vitro, the pre-irradiation step is routinely conducted without any background placed behind the substrate. In such instances, substrate with applied compositions is suspended (mounted) in the light beam, which creates conditions that are similar to the use of black background.

However, testing of sunscreen products efficacy in vivo is conducted on panelists with very light, light or intermediate skin using the specific selection guidelines: Fitzpatrick's classification and/or colorimetric ITA° value of skin. In particular, Fitzpatrick's classification for skin types is based on an individual's complexion and response to exposure to the sun: Type 1. Highly sun sensitive, always burns and never tans. Example—red hair with freckles; Type 2. Highly sun sensitive, burns easily and tans poorly. Example—fair skinned, fair haired Caucasians; Type 3. Sun sensitive, occasionally burns and slowly tans. Example—darker Caucasians; Type 4. Minimally sun sensitive, burns minimally and tans to moderate brown. Example—Mediterranean Caucasians; Type 5. Sun insensitive, rarely burns and tans well. Example—some Hispanics and some Blacks; Type 6. Sun insensitive, never burns and deeply pigmented. Example—darker Blacks. [Fitzpatrick T B. The validity and practicability of sun-reactive skin types I through VI, Archives Dermatol. 120, 869-871, 1988].

Colorimetric ITA° values and skin colour categories are defined by Chardon et al. using the CIE (1976) L*a*b* color space: Very Light—ITA° values>55°; Light—ITA° values from >41 to 55°; Intermediate—ITA° values from >28 to 41′; Tan (or Matt)—ITA° values from >10 to 28°; Brown—ITA° values from >−30 to 10°; Black—ITA° values<−30° where: ITA°=[Arc Tangent ((L*—50)/b*)]180/3.1416 [Chardon A, Crétois I, Hourseau C: Comparative colorimetric follow-up on humans of the tannings induced by cumulative exposures to UVB, UVA and UVB+A radiations. 16th IFSCC Congress, New-York, Preprint, vol 1, 51-70, 1990. &: Skin colour typology and suntanning pathways, Int. J. Cosm Scien. 125, 191-208, 1991].

Specifically, in vivo UVB efficacy testing of sunscreens requires only fair-skin subjects with skin types I, II, and III [FDA 21 CFR Parts 310, 352, 700, and 74. Sunscreen Drug Products For Over-The-Counter Human Use; Final Rule 1999] or with colorimetric ITA° value of skin that shall be greater than 28° [International Sun Protection Factor (SPF) Test Method. COLIPA Guidelines, 2006].

In vivo UVA efficacy testing of sunscreens requires panelists with skin types II and III only [FDA 21 CFR Parts 347 and 352. Sunscreen drug products for over-the-counter human use, proposed amendment of final monograph, Proposed Rules, Federal Register, §352.1, 72(165), 49070-49122 (2007)].

Thus, there is a contradiction that exists between the conditions of the in vitro test methods for evaluation of sunscreen formulation photo stability and efficacy that utilize pre-irradiation on black or similar to black background and in vivo efficacy tests that employ panelists with very light, light (or fair) and intermediate skin.

The impact of the skin color type and diffuse reflectance characteristics of various skin color types (or skin color categories) on sunscreen photo stability and efficacy parameters are not taken into account by these in vitro testing methodologies.

In addition, other existing in vitro methods for the determination of anti-ageing, ROS scavenging, antioxidant, photoprotective, UVA protective, photo stabilizing, and photosensitizing activities of topical ingredients and compositions that require pre-irradiation step do not take into account and ignore the impact of the skin color type and differences in diffuse reflectance characteristics of various skin color types (or skin color categories) on the respective activity parameters.

To overcome the deficiencies, irrelevancy, and contradictions associated with the prior art in vitro methods, the present invention provides in vitro methods for determination of activities and action mechanisms of topical ingredients and compositions on various skin color types permitting and providing relevancy to the in vivo conditions.

The present invention is directed to overcoming these and other deficiencies in the art.

### SUMMARY OF THE INVENTION

The present invention provides in vitro methods, apparatuses, and kits for determination of, inter alia, anti-ageing, photoprotective, sun protective, UVA/UVB protective, UVAI protective, photostabilizing, and photosensitizing activities and action mechanisms of topical ingredients and compositions on various skin color types. The methods of the present invention generally involve: (i) the use of substrates or well plates in conjunction with color backgrounds to approximate color and diffuse reflectance characteristics of various skin color types; (ii) pre-irradiation of the topical ingredients or compositions applied on the substrate or in the well plates that are placed on the color backgrounds; and (iii) determination of, inter alia, anti-ageing, photoprotective, sun protective, UVA/UVB protective, UVAI protective, photostabilizing, and photosensitizing activities and mechanisms of action of the topical ingredients or compositions by relevant experimental techniques or assays.

In one aspect, the present invention relates to an in vitro method for determining efficacy of a topical composition on a particular skin color type. This method involves Steps (a) through (d), as set forth below. Step (a) involves providing an artificial skin apparatus configured to approximate the color and diffuse reflectance characteristics of a predetermined human skin color type. The artificial skin apparatus includes an artificial skin substrate combined with a color background, with the color background correlating to the human skin color type. Step (b) involves applying a topical composition of interest to the artificial skin substrate of the artificial skin apparatus. Step (c) involves pre-irradiating the topical composition applied to the artificial skin substrate. Step (d) involves analyzing the pre-irradiated topical composition for at least one efficacy parameter.

In one embodiment, the in vitro method further includes Steps (e) and (f), as set forth below. Step (e) involves performing Steps (a) through (d) for the same topical composition at least one additional time using a different color background, thereby yielding efficacy parameters of the topical composition on a plurality of different color backgrounds. Step (f) involves comparing the efficacy parameters obtained from Step (e).

In another aspect, the present invention relates to an artificial skin apparatus for determining efficacy of a topical composition on a particular skin color type. The apparatus of the present invention includes an artificial skin substrate and a color background that correlates to a human skin color type. The artificial skin substrate and the color background are combined to yield an artificial skin apparatus that approximates the color and diffuse reflectance characteristics of a predetermined human skin color type.

In another aspect, the present invention relates to a kit for determining efficacy of a topical composition on a particular skin color type. The kit of the present invention includes an artificial skin apparatus according to the present invention and instructions for using the artificial skin apparatus to determine efficacy of a topical composition of interest on one or more different human skin color type.

Efficacy parameters and action mechanisms include, but are not limited to, anti-ageing, reactive oxygen species (ROS) scavenging, antioxidant, photo protective, sun protective, UVA/UVB protective, UVAI protective, photostabilizing, and photosensitizing activities. Topical ingredients and compositions include, but are not limited to, bioactive complexes, individual ingredients, sunscreen actives, or formulations for topical use. The pre-irradiation step of the in vitro method of the present invention can be conducted under natural or simulated sunlight or artificial irradiation conditions. Suitable substrates include, but are not limited to, artificial substrates replicating surface properties of human skin; profiled with the surface topography (roughness) of human skin; containing imprinted surface topography indentations approximating human skin; contoured to approximate human skin; roughened on product application side; and/or adapted for testing of the ultraviolet light absorbing and efficacy testing of topical compositions. Suitable wells include but are not limited to single well or multi-well plates. Suitable color backgrounds closely match and imitate color characteristics of various human skin color types. Color characteristics include, but are not limited to, Commission International de L'eclairage CIE L*a*b* values and the individual typology angles (ITA°). Experimental techniques include, but are not limited to, diffuse transmittance, diffuse reflectance in UV/VIS range, fluorescence in UV/VIS range, antioxidant and reactive oxygen species (ROS) scavenging assays based on fluorogenic, chromogenic or otherwise indicative probes, cell culture-based and skin equivalent-based assays.

The present invention provides an in vitro method that addresses the contradiction that exists between the conditions of the currently used in vitro test methods for evaluation of sunscreen formulation photostability and efficacy that utilize pre-irradiation on black or similar to black background and in vivo efficacy tests that employ panelists with very light, light (or fair), and intermediate skin.

The present invention also provides an in vitro method that, unlike the current in vitro testing methodologies, takes into account the impact of the skin color type and diffuse reflectance characteristics of various skin color types (or skin color categories) on sunscreen photostability and efficacy parameters.

The present invention also provides an in vitro method that, unlike the current in vitro testing methodologies that require a pre-irradiation step, takes into account the impact of the skin color type and differences in diffuse reflectance characteristics of various skin color types (or skin color categories) on the respective activity parameters in determining anti-ageing, ROS scavenging, antioxidant, photoprotective, UVA protective, photo stabilizing, photosensitizing activities of topical ingredients and compositions.

As set forth herein, the present invention provides in vitro methods for determination of activities and action mechanisms of topical ingredients and compositions on various skin color types, thereby permitting and providing relevancy to the corresponding in vivo conditions. Thus, the present invention provides, for the first time, in vitro methods, apparatuses, and kits that are effective in overcoming the deficiencies, irrelevancy, and contradictions associated with the prior art in vitro methods.

These and other objects, features, and advantages of this invention will become apparent from the following detailed description of the various aspects of the invention taken in conjunction with the accompanying drawings.

### BRIEF DESCRIPTION OF THE DRAWINGS

For the purpose of illustrating aspects of the present invention, there are depicted in the drawings certain embodiments of the invention. However, the invention is not limited to the precise arrangements and instrumentalities of the embodiments depicted in the drawings. Further, as provided, like reference numerals contained in the drawings are meant to identify similar or identical elements.

FIG. 1 is a black-and-white illustration of Leneta skin tone color chart 25 C backgrounds. Original colors, starting with the bottom band are: black, dark brown, light brown, yellow-beige, dark beige, light beige, white.

FIG. 2 is a graph showing UVAI-VIS (360 nm-740 nm) diffuse reflectance profiles of very light and light panelist skin and Vitro Skin® (N-19) placed on Leneta skin tone color chart 25 C backgrounds mimicking various skin color types. Solid lines are profiles of panelist skin and dashed lines are profiles of Vitro Skin® (N-19) placed on Leneta Chart 25 C backgrounds.

FIG. 3 is a graph showing diffuse reflectance profiles in UVAI-VIS (360 nm-740 nm) area of Vitro Skin® (N-19) with applied commercial sunscreen SPF 15 placed on Leneta Chart 25 C backgrounds mimicking various skin color types. Solid lines are before natural sunlight exposure and dashed lines—after exposure.

FIG. 4 is a graph showing diffuse reflectance profiles in UVAI (360-400 nm) area of Vitro Skin® (N-19) with and without applied commercial sunscreen SPF 15 placed on Leneta Chart 25 C backgrounds mimicking various skin color types—before and after exposure to the natural sunlight. The single solid line is profile of Vitro Skin® (N-19) placed on a specific color background, the double solid line is profile of Vitro Skin® (N-19) with applied commercial sunscreen SPF 15 placed on a specific color background, and dashed line is profile of Vitro Skin® (N-19) with applied commercial sunscreen SPF 15 placed on a specific color background after 10 MED exposure to natural sunlight.

FIG. 5 is an illustration showing interactions of sunlight with skin.

FIG. 6 is a graph showing similarity in UVAI-VIS (360 nm-740 nm) diffuse reflectance profiles of panelist skin of very light skin color type to Light Beige band of Leneta 25 C skin tone card covered with meniscus-forming layer of phospholipid liposome solution held in a polystyrene well. The dashed line is the solution-covered Light Beige band and solid line is very light panelist skin.

FIG. 7 is a graph showing irradiation dose-dependent increase in fluorescence of testing system with different probes. Darker round markers represent 2′,7′-dichlorofluorescin diacetate (DCFDA) and lighter square markers—Singlet Oxygen Sensor Green Reagent (SOSGR).

FIG. 8 is a graph showing fluorescence increase after 10 MED irradiation conducted on different color backgrounds. The darker columns represent Singlet Oxygen Sensor Green Reagent (SOSGR) and lighter columns—2′,7′-dichlorofluorescin diacetate (DCFDA).

FIG. 9 is a graph showing relationship between test article concentration and fluorescence increase after 10 MED irradiation with Singlet Oxygen Sensor Green Reagent (SOSGR) used as fluorescent probe.

### DETAILED DESCRIPTION OF THE INVENTION

The present invention is based, inter alia, on the novel and unexpected determination that the use of substrate/color background combinations that approximate color characteristics and diffuse reflectance parameters of various skin color types play a major role in the outcome of in vitro efficacy tests conducted to test topical compositions, where such tests involve a pre-irradiation step. Thus, one aspect of the present invention is the development of in vitro methods and apparatuses that take into account the novel and unexpected determination that using color backgrounds in conjunction with an appropriate substrate creates useful tools and methodologies that effectively approximate in vivo activities of compositions on particular skin color types.

As set forth in more detail herein, by providing in vitro methods for determining efficacy and mechanisms of action for a topical composition of interest, the present invention is effective to provide relevancy to various in vivo conditions in response to or relation to a topical composition of interest. The present invention is useful for analyzing all types of topical compositions for application to human skin. Further, the present invention is useful for analyzing all types of efficacies and mechanisms of action for any and all such topical compositions. While not intending to limit the present invention, particular efficacies that can be measured for a topical composition on various skin color types can include, without limitation, anti-ageing, photoprotective, sun protective, UVA/UVB protective, UVAI protective, photostabilizing, and photosensitizing activities. Further, the present invention is also useful for determining the action mechanisms of topical compositions on various skin color types.

As used herein, the terms “mechanism of action” and “action mechanism” refer to any mechanism triggered by application of a topical composition on human skin or on an artificial skin substrate of the present invention. While not intending to limit the present invention, in one embodiment, a mechanism of action relates to the fact that the higher diffuse reflectance of lighter skin color types, especially in UVA-VIS area, increases the probability for more photons to be reflected—not absorbed—and to react with components of the skin, topical ingredients, and compositions distributed within upper layers of a substrate or skin and to participate in the photosensitization processes and reactive oxygen species (ROS) generation, which will further contribute to the increase in photoinstability, and to the oxidative damage processes. Presently, ROS generated by UV/VIS light—related mechanisms of photoinstability of sunscreen actives (avobenzone, octinoxate, etc.) in the upper layers of very light to intermediate skin are not addressed by prior art in vitro methods, in which black (dark) background or equivalents are used during pre-irradiation step. Apparently, the intensities of ROS interactions with sunscreen actives, other photounstable, or photoreactive compositions and skin constituents in upper layers of skin (stratum corneum and epidermis) are different on light and dark skin color types. On the dark (black) skin, due to the lower diffuse reflectance in UV-VIS range, more photons are absorbed by basal cells and melanocytes located in deeper layers of skin, which potentially can lead to and also explain the differences in damage to these particular cells depending on skin color type. The present invention is effective for use in studying these and other mechanisms of action.

Generally, in one aspect, the present invention provides an in vitro method that involves the following: (i) the utilization of substrates or well plates in conjunction with color backgrounds with the properties of the resulting combination of substrate placed on the background approximating color and diffuse reflectance characteristics of various skin color types; (ii) pre-irradiation of the topical compositions of interest applied on the substrate or in the well plates that are placed on the color backgrounds; and (iii) determination of anti-ageing, photoprotective, sun protective, UVA/UVB protective, UVAI protective, photostabilizing, and photosensitizing activities and mechanisms of action of the topical compositions by relevant experimental technique or assay.

Various aspects of the present invention are set forth in this paragraph. However, certain of these aspects are repeated and expanded upon in other parts of the present disclosure. Efficacy parameters and action mechanisms include, but are not limited to, anti-ageing, reactive oxygen species (ROS) scavenging, antioxidant, photo protective, sun protective, UVA/UVB protective, UVAI protective, photostabilizing, and photosensitizing activities. Topical compositions include but are not limited to bioactive complexes, individual ingredients, sunscreen actives, or formulations for topical use. The pre-irradiation step is conducted under natural or simulated sunlight or artificial irradiation conditions. Suitable substrates include but are not limited to: artificial substrates replicating surface properties of human skin; profiled with the surface topography (roughness) of human skin; containing imprinted surface topography indentations approximating human skin; contoured to approximate human skin; roughened on product application side; adapted for testing of the ultraviolet light absorbing and efficacy testing of topical compositions. Suitable wells include but are not limited to single well or multi-well plates. Suitable color backgrounds closely match and imitate color characteristics of various human skin color types. Color characteristics include but are not limited to Commission International de L′eclairage CIE L*a*b* values and the individual typology angles)(ITA°. Experimental techniques include but are not limited to diffuse transmittance, diffuse reflectance in UV/VIS range, fluorescence in UV/VIS range, fluorogenic probes—based antioxidant and reactive oxygen species (ROS) scavenging assays, cell culture-based and skin equivalent-based assays.

As presented herein, the present invention provides an in vitro method that is suitable for use with available assays that are conducted on cell cultures and skin equivalents that require a pre-irradiation step. However, unlike the assays in the prior art, the in vitro method of the present invention includes steps that appreciate the potential impact of skin tone (or even background color) on the relevant activities/properties of the topical compositions. For example, while cell cultures and skin equivalents are semi-transparent, unlike the in vitro method of the present invention, pre-irradiation conditions used in the current methods do not specify the background color on which the wells are placed during pre-irradiation.

In one aspect, the present invention relates to an in vitro method for determining efficacy of a topical composition on a particular skin color type. The topical composition can be bioactive complexes, a single ingredient, a mixture of ingredients, and/or a formulation for topical use. This method involves Steps (a) through (d), as set forth below. Step (a) involves providing an artificial skin apparatus configured to approximate the color and diffuse reflectance characteristics of a predetermined human skin color type. The artificial skin apparatus includes an artificial skin substrate combined with a color background, with the color background correlating to the human skin color type. Step (b) involves applying a topical composition of interest to the artificial skin substrate of the artificial skin apparatus. Step (c) involves pre-irradiating the topical composition applied to the artificial skin substrate. The pre-irradiation of Step (c) can be conducted under natural or simulated sunlight or artificial irradiation conditions. Step (d) involves analyzing the pre-irradiated topical composition for at least one efficacy parameter. Various aspects of Steps (a) through (d) are further described herein below.

In one embodiment of the in vitro method of the present invention, the artificial skin substrate is configured to have properties that include, without limitation, surface properties that replicate those of human skin, surface topography (roughness) corresponding to that of human skin, contours that approximate human skin, roughened surface, and/or properties adapted for testing of the ultraviolet light absorbing and efficacy testing of topical compositions.

Suitable examples of particular artificial skin substrates for use in the present invention can include, without limitation, VITRO SKIN® (N-19), PMMA-based substrates, quartz-based substrates, polymer films having a thickness of about 100-1,000 μm and exhibiting at least 10% transmission of light having a wavelength of about 280-450 nm, polypropylene-based substrates, and the like.

Further, the artificial skin substrate can be used in various forms, including, for example, in the form of a single well or a plurality of wells. In a particular embodiment, the artificial skin substrate can be in the form of a single well or a plurality of wells used in conjunction with a layer of phospholipid liposomes, essential constituents of biological membranes, or the like, and/or with a cell culture or skin equivalent.

The color background for use in the present invention can be a material that correlates to any human skin color type, including, without limitation, very light, light, intermediate, tan, brown, and black human skin color types.

A suitable color background can be a material that corresponds to a color of a Leneta Chart 25 C, including the color white, light beige, dark beige, yellow beige, light brown, dark brown, or black.

Other suitable color backgrounds include, but are not limited to, IMS Human Skin Tone Chart (IMS, Inc. Milford, Conn., US).

In a particular embodiment, the artificial skin substrate can be VITRO SKIN® (N-19) combined with a color background from the Leneta Skin tone color chart 25 C. This particular combination of artificial skin substrate and color background effectively approximates color characteristics, ITA° values, and diffuse reflectance parameters of various human skin color types.

Other suitable artificial skin substrates for use in the present invention include, but are not limited to, those substrates described below.

For example, one suitable artificial skin substrate can include, without limitation, the substrate described in U.S. Pat. No. 7,004,969 (Shiseido Company, Ltd.), which is hereby incorporated by reference herein in its entirety. This substrate having a thickness of about 100 to 1,000 μm is prepared from a polymer which, when formed into a thin film having a thickness of about 100-1,000 μm, exhibits a percent transmission of light having a wavelength of about 450-280 nm of at least about 10%. In this substrate, grooves, which imitate furrows, are provided on one surface.

Another suitable artificial skin substrate can include, without limitation, PMMA-based substrates, PMMA HD2 or HD6 with 2 or 6 micron roughness, respectively. Examples of such PMMA-based substrates can be readily determined by those of ordinary skill in the art, including, without limitation, from resources available via the Internet [see, e.g., www.biblioscreen.helioscreen.fr/Documents%20helioscreen/LivretHelioplatesang.pdf, which is hereby incorporated by reference herein in its entirety].

Another suitable artificial skin substrate can include, without limitation, Quartz-based substrates. In a particular embodiment, the Quartz-based substrate is roughened on the application side. In another particular embodiment, the Quartz-based substrate can include, without limitation, a Quartz-based MimSkin® v.1.0 substrate and the like [see, e.g., www.aptf.com.au/mimskin, which is hereby incorporated by reference herein in its entirety].

Another suitable artificial skin substrate can include, without limitation, the artificial substrate described in International Patent Application No. WO/2008/113109 A1, which is hereby incorporated by reference herein in its entirety. This substrate is adapted for use in testing of performance factors of topical lotions or creams, the substrate comprising one or more layers of polypropylene tape bonded to a polypropylene film, wherein the polypropylene tape has imprinted surface topography indentations therein.

The in vitro method of the present invention is effective for determining various efficacy parameters and mechanisms of action of the topical compositions of interest on one or more type of human skin color. After the pre-irradiation step, various experimental techniques and assays can be used to test and determine any and all efficacy parameters commonly measured in the relevant field.

In one embodiment, the at least one efficacy parameter is effective to measure an activity that includes, but is not limited to, anti-ageing activity, photoprotective activity, sun protective activity, UVA/UVB protective activity, UVAI protective activity, photostabilizing activity, and photosensitizing activity. Suitable techniques and assays to measure these activities are well known in the art, and are contemplated by the present invention. In a particular embodiment, suitable assays for use in determining the at least one efficacy parameter can include, without limitation, a diffuse transmittance assay, a diffuse reflectance in UV/VIS range assay, a fluorescence in UV/VIS range assay, a free radical assay, an antioxidant assay, a reactive oxygen species (ROS) assay, and the like. Performing these and other suitable techniques and assays to measure the at least one efficacy parameter are well known in the art, and are contemplated by the present invention. For example, in one embodiment, the at least one efficacy parameter is measured by determining products of irradiation using, for example, spectrophotometric, chromatographic, mass spectroscopy, nuclear magnetic resonance, and/or electron paramagnetic resonance techniques. Performing these and other suitable techniques and assays to measure the at least one efficacy parameter are well known in the art, and are contemplated by the present invention.

In one embodiment, the in vitro method further includes Steps (e) and (f), as set forth below.

Step (e) involves performing Steps (a) through (d) for the same topical composition at least one additional time using a different color background, thereby yielding efficacy parameters of the topical composition on a plurality of different color backgrounds.

Step (f) involves comparing the efficacy parameters obtained from Step (e).

In another aspect, the present invention relates to an artificial skin apparatus for determining efficacy of a topical composition on a particular skin color type. The apparatus of the present invention includes an artificial skin substrate and a color background that correlates to a human skin color type. The artificial skin substrate and the color background are combined to yield an artificial skin apparatus that approximates the color and diffuse reflectance characteristics of a predetermined human skin color type.

The various artificial skin substrates and color backgrounds of the artificial skin apparatus of the present invention are as described herein with respect to the use of the apparatus in the in vitro method of the present invention. Thus, the various artificial skin substrates and color backgrounds of the artificial skin apparatus of the present invention are not duplicated here.

In another aspect, the present invention relates to a kit for determining efficacy of a topical composition on a particular skin color type. The kit of the present invention includes an artificial skin apparatus according of the present invention and instructions for using the artificial skin apparatus to determine efficacy of a topical composition of interest on one or more different human skin color type.

### EXAMPLES

The following examples are intended to illustrate particular embodiments of the present invention, but are by no means intended to limit the scope of the present invention.

### Example 1

### Evaluation of Sunscreen's Efficacy and Photostability

In vitro studies of sunscreen's efficacy and photostability were conducted on substrates mentioned in Table I and also on collagen-containing substrate Vitro Skin® (N-19)—under natural sun exposure and also using full spectrum solar light simulator according to the methodologies described in Table I. During the pre-irradiation step substrates with applied commercial sunscreen products were placed on the color backgrounds of Skin tone color chart 25 C from The Leneta Company (Mahwah, N.J., US) that mimics various skin color types. This chart was developed based on 1976 Commission International de L'eclairage CIE L*a*b* values of skin tones measured on numerous volunteers and possesses excellent shade uniformity, color density, reproducibility and non-fluorescence [Gabriel E. Uzunian and Olga V. Dueva. The Impact of Skin Tone on the Color Generated by Effect Pigments. J. Cosmet. Sci., 52, 419-420 (2001), which is hereby incorporated by reference herein in its entirety].

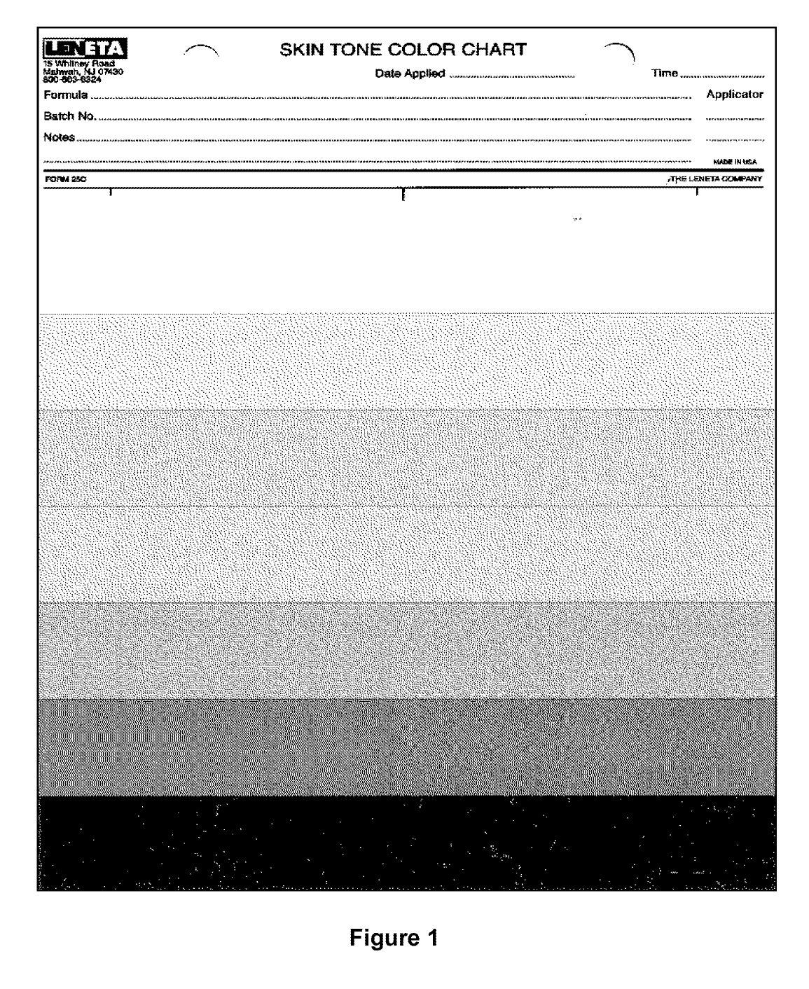

The Leneta skin tone color chart 25 C backgrounds are presented in FIG. 1, which is a black-and-white illustration of Leneta skin tone color chart 25 C backgrounds. The original colors, starting with the bottom band are: black, dark brown, light brown, yellow-beige, dark beige, light beige, white.

The Leneta skin tone color chart 25 C backgrounds were covered with Vitro Skin® (N-19) substrate and their respective L*a*b* values measured on Konica Minolta CM 2600d Spectrophotometer (10° observer, primary illuminant D65 with UV setting 100% Full; Specular Component excluded). The individual typology angles (ITA°) for the resulting combination of Vitro Skin® (N-19) substrate placed on backgrounds of skin tone color chart were calculated based on the following equation: ITA°=[Arc Tangent ((L*−50)/b*)]180/3.1416 and compared with ITA° values of different human skin color types: Very Light>55°; Light>41 to 55°; Intermediate>28 to 41°; Tan (Matt)>10 to 28°; Brown>−30° to 10°; Black<−30° [Chardon A, Crétois I, Hourseau C: Comparative colorimetric follow-up on humans of the tannings induced by cumulative exposures to UVB, UVA and UVB+A radiations. 16th IFSCC Congress, New-York, Preprint, vol 1, 51-70, 1990 & Skin colour typology and suntanning pathways, Int. J. Cosm Scien. 125, 191-208, 1991, which are hereby incorporated by reference herein in their entirety].

ITA° values of the resulting combination of Vitro Skin® (N-19) substrate placed on the backgrounds of skin tone color chart are as follows: 84° for white; 61° for light beige; 45° for dark beige; 19° for light brown; −55° for dark brown; and −89° for black.

Thus, ITA° values of light beige, dark beige, light brown and dark brown backgrounds covered with Vitro Skin® (N-19) substrate correspond to ITA° of very light, light, tan and black skin color types, respectively.

Diffuse reflectance of human panelists' light or very light skin types and Vitro Skin® (N-19) placed on the Leneta skin tone color chart 25 C backgrounds was measured in UVAI-VIS area (360-740 nm) on Konica Minolta CM 2600d Spectrophotometer (10° observer, primary illuminant D65 with UV setting 100% Full; Specular Component excluded).

Data are presented on FIG. 1, which illustrates similarity in UVAI-VIS (360 nm-740 nm) diffuse reflectance profiles of very light and light panelist skin to Vitro Skin® (N-19) placed on Leneta Chart 25 C background mimicking various skin color types.

Diffuse reflectance spectra of Vitro Skin® (N-19) placed on the Leneta skin tone color chart 25 C backgrounds were compared with the diffuse reflectance spectra of various color types of human skin measured in vivo and also with the relevant data previously reported in the literature.

For example, it was reported that the diffuse reflectance of Caucasian skin in UV-VIS area is about 3 times higher compared with black skin [R. Rox Anderson and John Parrish. Optics of Human Skin. Journal Investigative Dermatology, 77, 13-19 (1981), which is hereby incorporated by reference herein in its entirety].

It was also reported that the diffuse reflectance in UV-VIS area of skin type II is about 2.3 times higher than that of skin type IV. In addition, the diffuse reflectance in UV-VIS area of skin type II is about 1.4 times higher than that of skin type III. [Kristian P. Nielsen et. al. The optics of human skin: Aspects important for human health. In Solar Radiation and Human Health; Espen Bjertness, Editor. Oslo: the Norwegian Academy of Science and Letters, 35-46 (2008), which is hereby incorporated by reference herein in its entirety].

A comparison of the diffuse reflectance in UVA/VIS area of Vitro Skin® (N-19) placed on the Leneta Chart 25 C backgrounds with the diffuse reflectance of various color types of human skin is presented in Table II.

TABLE II
A comparison of the diffuse reflectance in UVA/VIS area of Vitro
Skin ® (N-19) placed on the Leneta Chart 25C backgrounds with
the diffuse reflectance of various color types of human skin

| | | | Diffuse Reflectance Ratios of | | |
|---|---|---|---|---|---|
| Diffuse Reflectance Ratios | | | Vitro Skin ® (N-19) | | |
| among Human Skin | | | placed on the Leneta skin tone | | |
| Color Types | | | color chart 25C backgrounds | | |
| | Skin | Skin | | Light | |
| | type II/ | type II/ | | Beige/ | Dark Beige/ |
| Caucasian/ | Skin | Skin Type | Dark Beige/ | Light | Light |
| Black | IV | Type III | Dark Brown | Brown | Brown |
| 3.1 | 2.3 | 1.4 | 2.9 | 2.1 | 1.5 |

The diffuse reflectance parameters of Vitro Skin® (N-19) placed on light beige, dark beige, light brown and dark brown backgrounds of the Leneta Chart 25 C correlates well with these parameters of very light, light, tan and black human skin color types, respectively.

Based on these data we have concluded that for sunscreen's efficacy and photostability evaluations Vitro Skin® (N-19) is a preferred substrate and the Leneta Skin tone color chart 25 C is a preferred color background; the resulting combination of preferred substrate and background effectively approximates color characteristics, ITA° values and diffuse reflectance parameters of various human skin color types.

### Example 2

### In Vitro/In Vivo Tests of Sunscreen's Photostability and Efficacy Under Natural Sunlight Exposure

A commercial sunscreen SPF 15 containing UVB/UVA absorbers (sunscreen actives): 3% Avobenzone, 7.5% Octinoxate and 2% Octisalate (Aveeno Active Naturals Positively Radiant Daily Moistrizer SPF 15 UVA/UVB sunscreen, Lot 0050C, Exp. 2012/01) was utilized as test article. Avobenzone (or 4-tert-butyl-4-methoxydibenzoylmethane—BMDBM) is one of the most important UVA filters in commerce today. Unfortunately, this molecule is photo-unstable; it has been reported to fragment when exposed to UV radiation into reactive species. Avobenzone reacts with other molecules including octinoxate (or ethylhexyl methoxy cinnamate) to yield photoadducts. Numerous attempts to photostabilize avobenzone have been introduced, including encapsulated organic sunscreens, microspheres, ROS quenchers, triplet-triplet quenchers, singlet-singlet quenchers [Nadim A. Shaath. Ultraviolet filters. Photochem. Photobiol. Sci., 2010, 9, 464-469, which is hereby incorporated by reference herein in its entirety].

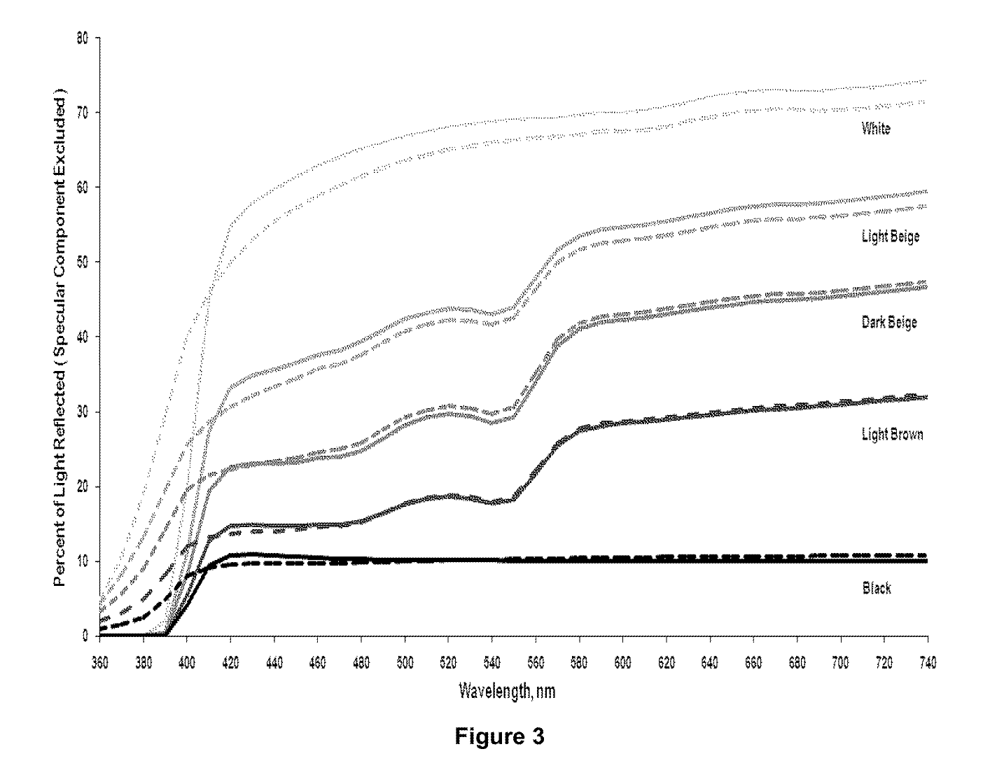

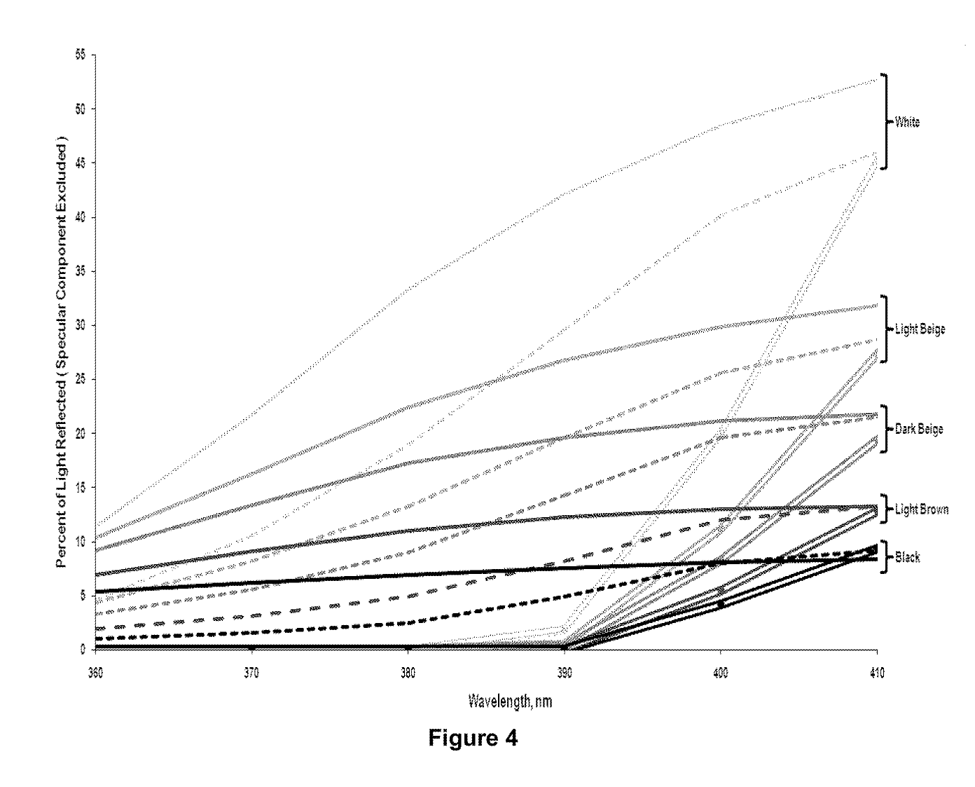

SPF 15 sunscreen was applied on Vitro Skin® (N-19) substrates (Lot 9202); application dose was 2 mg/sq. cm; application technique was described in [Olga V. Dueva-Koganov et. al. Addressing Technical Challenges Associated with FDA Proposed Rules for UVA In Vitro Testing Procedure. J. Cosmet. Sci., 60, 587-598 (2009), which is hereby incorporated by reference herein in its entirety]; test articles were placed on different backgrounds of the Leneta Skin tone color chart 25 C and exposed to natural sunlight. All natural sunlight exposure in vitro and in vivo photostability and efficacy tests were performed on the same day (Mar. 23, 2010 in Playa del Carmen, Mexico, Latitude: 20° 38′ 6.36″ N; Longitude: 87° 4′ 49.59″ W; from 10 AM to 2 PM). Cumulative irradiation dose was about 10 Minimal Erythemal Doses (MEDs) determined with PMA2100 Radiometer and PMA2101 Detector (all from SolarLight Company, PA); this dose is consistent with one required by the FDA Proposed rules for SPF 15 sunscreen [Food and Drug Administration 21 CFR Parts 347 and 352. Sunscreen drug products for over-the-counter human use, proposed amendment of final monograph, Proposed Rules, Federal Register, §352.1, 72(165), 49070-49122 (2007), which is hereby incorporated by reference herein in its entirety]. Temperature of the test articles during natural sunlight exposure has not exceeded 40 deg. C. Diffuse reflectance and diffuse absorbance measurements of substrates with applied sunscreen were conducted before and after natural sunlight exposure; experimental data are presented on FIGS. 3-4 and in the Table III.

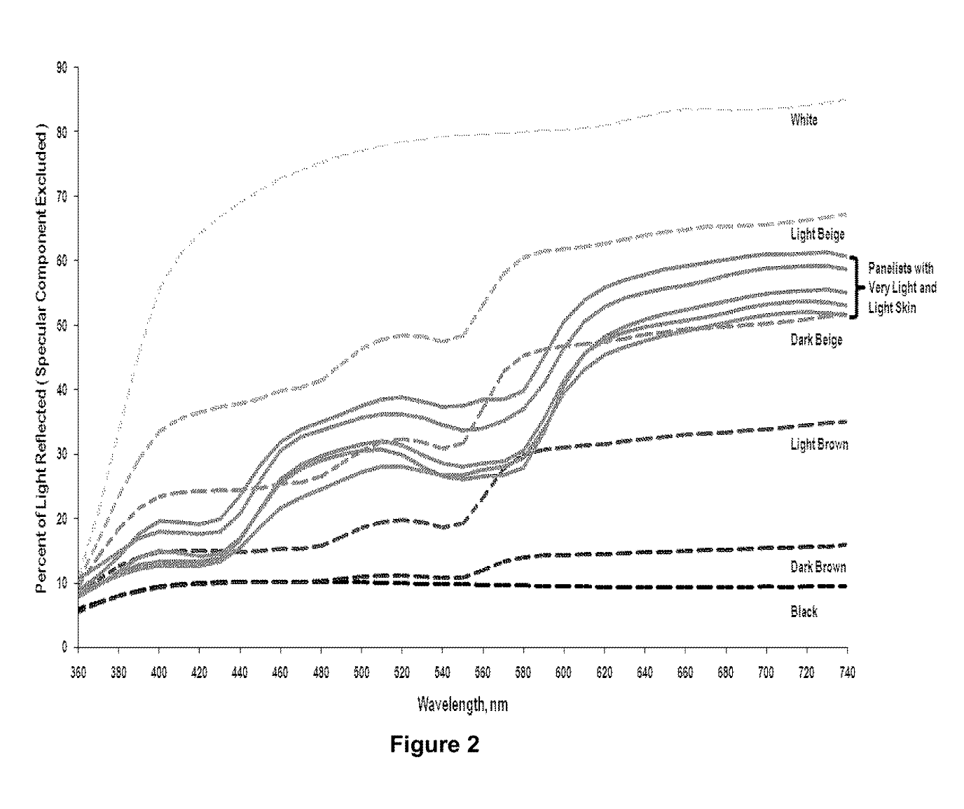

Data presented in FIG. 3 indicate that after sunscreen SPF 15 was applied on the substrate, its diffuse reflectance in UVAI area was reduced on all backgrounds—when compared with the diffuse reflectance of untreated substrates placed on the same backgrounds presented in FIG. 2. Such initial decrease of diffuse reflectance in this area was expected due to the presence of sunscreen actives in the formulation.

FIG. 3 also shows that the changes in diffuse reflectance in VIS (400-740 nm) area after sunscreen application compared to untreated substrate (blank) were insignificant on all color backgrounds.

At the same time, the degree of change (an increase) in diffuse reflectance of sunscreen in UVAI area after natural sunlight exposure significantly varied depending on the background color. The increase in the diffuse reflectance in UVAI (360-400 nm) area after natural sunlight exposure corresponds to the degree of the UVAI photoinstability of the formulation and reflects the loss of UVAI protection efficacy—a larger increase corresponds to lower UVAI protection remaining.

In VIS area (400-740 nm) the changes in the diffuse reflectance spectra after irradiation were either less pronounced or there was no change at all—regardless of the background color.

FIG. 4 represents diffuse reflectance profiles in UVAI (360-400 nm) area of Vitro Skin® (N-19) with and without applied commercial sunscreen SPF 15 placed on Leneta Chart 25 C backgrounds mimicking various skin color types—before and after 10 MED exposure to the natural sunlight.

Clearly, UVA photostability of a sunscreen is significantly influenced by a background color, on which substrate is placed and the diffuse reflectance of substrate/background combination, especially in UVA-VIS area. Sunscreen SPF 15 was the most UVAI photostable when it was pre-irradiated on a black background; its photostability has slightly decreased on light brown and significantly decreased on dark beige followed by more significant decrease on light beige background.

A comparison of substrate reflectance spectra in UVAI area—initial, after sunscreen application, and after irradiation shows that the remaining UVAI protection on black background was about 65.5%, on light brown—59%, on dark beige—51%, on light beige—49%.

Thus, on black background the photostability of a sunscreen was about 34-30% higher than on light beige or dark beige backgrounds, respectively.

Diffuse reflectance in vitro data were confirmed by diffuse transmittance in vitro measurements of the same test articles using Labsphere UV 2000S Transmittance Analyzer with an integrating sphere and photodetector providing a continuous emission spectrum from 290-400 nm with sufficient illumination at each wavelength, but not in excess of 0.2 J/cm2. The dynamic range of this instrument (290 to 400 nm) is 2.7 or more Absorbance units.

Sunscreen efficacy parameters after natural sunlight exposure (10 MED) were measured according to the FDA Proposed Rules (2007) guidelines and are presented in Table III.

TABLE III
Sunscreen efficacy parameters after natural sunlight exposure
measured via diffuse transmittance

| | Sunscreen UVA Efficacy/Photostability | | |
|---|---|---|---|
| The Leneta Skin | Parameters measured according the | | |
| Tone Color Chart | FDA Proposed Rules (2007) guidelines | | |
| 25C | UVAI/UV Ratio in | | Critical |
| Background | vitro | Category | Wavelength, nm |
| White | 0.39 | Low | 360 |
| Light Beige | 0.39 | Low | 362 |
| Dark beige | 0.43 | Medium | 365 |
| Light Brown | 0.47 | Medium | 367 |
| Black | 0.54 | Medium | 370 |

Higher UVAI/UV ratio and Critical Wavelength values indicate better UVA efficacy and photostability of the sunscreen formulation.

On the black background sunscreen's photostability determined by UVAI/UV ratio was about 38 to 25% higher than on light beige and dark beige backgrounds, respectively.

Similar trends in sunscreen UVA efficacy/photostability being affected by the background color were observed when PMMA HD2, PMMA HD6 or quartz based substrates were utilized under these test conditions.

In vitro findings obtained on dark beige background were confirmed by the in vivo data obtained on several volunteers with light skin type by the comparison of diffuse reflectance measurements in UVAI-VIS area (360-740 nm) on Minolta CM 2600d Spectrophotometer (10° observer, primary illuminant D65 with UV setting 100% Full; Specular Component excluded) of test sites (volar aspects of panelists forearms) conducted before sunscreen SPF 15 application, after sunscreen application (application dose 2 mg/sq. cm)—before natural sun exposure and after natural sun exposure (10 MEDs).

### Example 3

### Sunscreen Efficacy and Photostability Parameters Measured According to COLIPA (2009) Guidelines after Simulated Sunlight Irradiation

A commercial sunscreen SPF 15 described in Example 2 was applied on PMMA HD2 substrates (application dose was 0.75 mg/sq. cm); test articles were placed on different backgrounds of the Leneta skin tone color chart 25 C and subjected to simulated sunlight exposure using 16S-300-002 Solar Simulator (SolarLight Company, PA) that produces full spectrum sunlight (Air Mass, AM 1.5) with a vertical beam adapter redirecting the light beam to point downward. The spot size is 3.3 cm with variable 1 to 4 sun maximum output intensity. XPS 400 was used as a precision current source for 16S-300-002. Irradiation intensity and irradiation doses for each substrate measured with PMA2100 Radiometer and PMA2101 Detector (all from SolarLight Company, PA) were in compliance with 2009 COLIPA guidelines. For temperature control to prevent any overheating during irradiation, a Peltier-cooled surface was used (Torrey Pines Scientific SC25 with microplate holder attachment). Diffuse absorbance measurements of substrates with applied sunscreen were conducted before and after irradiation; the results are presented in Table IV.

TABLE IV
Sunscreen efficacy and photostability parameters after simulated sunlight
irradiation measured according to COLIPA (2009) guidelines

| The Leneta | | | | | | | |
|---|---|---|---|---|---|---|---|
| Color Chart | | UVAPF | Irradiation | UVAPF | Ratio | Critical | |
| 25C | SPF in | before | Dose, J/cm² | after | (UVAPF/ | Wavelength, | UVA |
| Background | vitro | irradiation | UVA | irradiation | SPF in vivo) | nm | logo |
| White | 13 | 9 | 10.42 | 4 | 0.26 | 360 | Not |
| | | | | | | | allowed |
| Light Beige | 14 | 9 | 9.58 | 4 | 0.26 | 361 | Not |
| | | | | | | | allowed |
| Black | 13 | 9 | 10.42 | 5 | 0.33 | 371 | Allowed |

Thus, the use of black background during pre-irradiation as required by COLIPA (2009) would allow the UVA logo claim, other backgrounds will not.

This proves that when black background is used it provides unrealistic and irrelevant test conditions and SPF 15 sunscreen's photostability determined on the black background is overestimated.

This can result in higher photostability values reported, which will be unsustainable outside artificially favorable laboratory in vitro conditions—especially given the fact that sunscreen's efficacy and photostability is more critical for very light to intermediate skin types, not for tan or black skin types because only very light to intermediate skin types are considered as photoreactive skin types and are employed in SPF, PPD-UVA-PF in vivo efficacy studies.

The existing methodologies for the determination of sunscreen photostability in vitro do not take into the account the impact of the background skin color type and differences in diffuse reflectance in UVA-VIS area associated with various skin types on sunscreen's photo stability.

We have unexpectedly found that the background skin color type and differences in diffuse reflectance associated with various skin types produce a large impact on sun exposure related processes that are happening to externally (topically) applied compositions, formulations, sunscreen products, etc. on the surface of the substrate.

We have also unexpectedly found that higher diffuse reflectance of very light and light skin types in UVA-VIS area is associated with increase of the photoinstability of topically applied sunscreen actives (avobenzone, octinoxate, etc) and other potentially photounstable or photoliable molecules and compositions.

This finding represents a mechanism of photoinstability that was not described or appreciated before, especially taking into the account that dark (black) background is widely used in the sunscreen research and development during pre-irradiation step in vitro specifically to minimize reflection of UV radiation back through the sample [P. J. Matts et. al. The COLIPA in vitro UVA method: a standard and reproducible measure of sunscreen UVA protection. International Journal of Cosmetic Science 2010, 32, 35-46, which is hereby incorporated by reference herein in its entirety], which simultaneously minimizes the reflectance of UV-VIS light.

This mechanism of photoinstability is taking place during exposure (irradiation) to natural sun, simulated full spectrum (UV-VIS) sun and to the artificial light sources with UV-VIS or UVA-VIS components present and is more pronounced on very light, light or intermediate skin color types or on the backgrounds that mimic color characteristics determined by ITA° values or CIE L*a*b*values and diffuse reflectance parameters of these human skin color types.

This mechanism of photoinstability is taking place to a lesser extent in the following situations: when in vitro photostability studies are conducted under artificial irradiation conditions similar to those during SPF in vivo testing under UVB/UVA (290-400 nm) or PPD UVA-PF in vivo testing under UVA (320-400 nm); when UVA-VIS contribution to the irradiation spectra is altered and minimized, or UVB-VIS contributions are minimized, respectively; when the optical density of the system is very high, giving increase to a self-protection effect of the system or sunscreen film; when test formulation has very high SPF value and is sufficiently photostabilized.

This mechanism of photoinstability can be addressed by the utilization of the antioxidants and photostabilizers with high specific efficacy and confirmed absence of pro-oxidant activity at the wide concentration range in test models mimicking end usage conditions—topical application on various skin color types backgrounds followed by exposure (irradiation) to simulated full (UV-VIS) spectrum sun, natural sun, or to the artificial light sources.

Our findings have increased an understanding of the effects of natural sunlight or simulated radiation on the processes that are taking place on substrates depending on the background color and on different skin color types/phototypes and suggest the following: in vitro testing methodologies used for the development of effective anti-ageing and sunscreen products should appreciate and address the mechanism of photoinstability described in present invention; formulating approaches and in vitro testing methodologies used for the development of photostable sunscreen and effective anti-ageing products should be customized for different skin color types and reflect end-use conditions.

One of the plausible theoretical explanations for our findings includes but is not limited to the implications of the first law of photochemistry (Grotthus-Draper Law), stating that photon must be absorbed by an atom or molecule in order to initiate physico-chemical process.

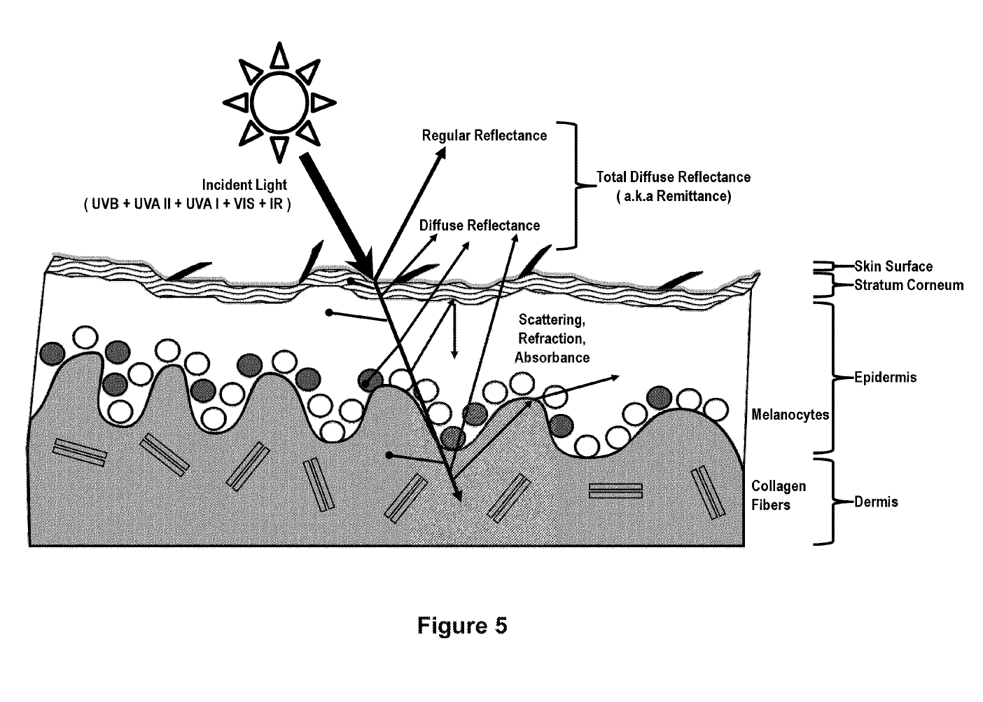

Upon interaction with skin or substrate, sunlight can be reflected, scattered or absorbed as shown at the diagram of sunlight interactions with human skin presented in FIG. 5. The diffuse reflectance in UVA-VIS area of very light to intermediate skin color types is about 3+ to 2 times higher compared with darker (black) skin color types (see Table II). Thus, higher diffuse reflectance of lighter skin color types especially in UVA-VIS area increases the probability for more photons to be reflected—not absorbed and to react with components of skin, topical ingredients and compositions distributed within upper layers of a substrate or skin and to participate in the photosensitization processes and reactive oxygen species (ROS) generation, which will further contribute to the increase in photoinstability, and to the oxidative damage processes.

ROS generated by UV/VIS light—related mechanisms of photoinstability of sunscreen actives (avobenzone, octinoxate, etc.) in the upper layers of very light to intermediate skin are not addressed by prior art in vitro in which when black (dark) background or equivalents are used during pre-irradiation step. Apparently intensities of ROS interactions with sunscreen actives and ingredients of topical products and skin constituents in upper layers of skin (stratum corneum and epidermis) are different on light and dark skin color types. Due to the lower diffuse reflectance in UV-VIS for dark (black) skin, more photons are absorbed by basal cells and melanocytes located in deeper layers of skin, which potentially can lead to and also explain the differences in damage to these particular cells depending on skin color type.

For example, such differences were observed at human research focusing on reactive oxygen species formation at basal cell level in the epidermis: in resting (basal) skin samples, there were significantly higher levels of ROS in the facial skin of dark complexioned subjects compared to the light complexioned subjects; skin oxidative stress responses to external aggression from solar simulator are greater in dark complexioned individual than light complexioned individuals [Michelle Garay et al. Skin oxidative stress responses to external aggression are greater in dark complexioned individuals than light complexioned individuals. Journal of the American Academy of Dermatology, 1 Mar. 2009, volume 60 issue 3 Page AB28, which is hereby incorporated by reference herein in its entirety]. Our findings help to explain the high standard deviations reported when sunscreens were tested for their UVA efficacy and photostability in vivo using panelists with skin types II-IV [Eduardo Ruvolo Jr. et. al. Diffuse reflectance spectroscopy for ultraviolet A protection factor measurement: correlation studies between in vitro and in vivo measurements. Photodermatology, Photoimmunology & Photomedicine 2009, 25, 298-304, which is hereby incorporated by reference herein in its entirety].

### Example 4

### In Vitro Methods to Evaluate Antioxidant, Anti-Ageing Activities and Action Mechanisms of the Compositions on Various Skin Color Types

The existing methodologies for the determination of anti-oxidant and anti-ageing activities in vitro do not take into the account the significant impact of the background skin color type and differences in diffuse reflectance in UV-VIS area associated with various skin color types on the test outcome.

An in vitro system was developed to model a common mechanism of sunlight damage to the various skin color types and in particular the stratum corneum by simulated sunlight irradiation—induced radical and oxidative damage products generation. It is composed of a buffered phospholipid liposome solution serving as the reaction medium and a substrate for production of radicals and oxidative damage products, a solution of fluorogenic probe sensitive to products of sunlight damage serving to make the damage quantifiable, and a test article or vehicle control to assess efficacy of the test article in preventing and mitigating sunlight induced damage and potential for undesirable pro-oxidant properties. This system was tested in black 96-well microtiter plates with transparent polystyrene bottoms (Corning 3651).

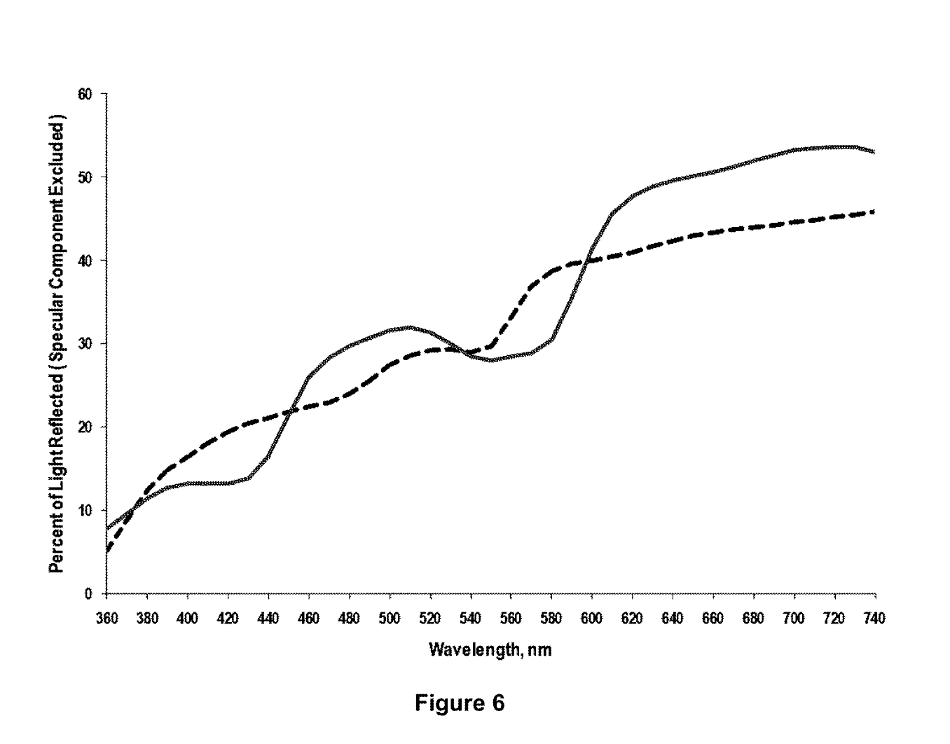

FIG. 6 shows similarity in UVAI-VIS (360 nm-740 nm) diffuse reflectance profiles of panelist skin of very light color type to Light Beige band of Leneta 25 C skin tone card covered with meniscus-forming layer of phospholipid liposome solution-based test system held in a polystyrene well.

To ensure consistent temperature in all microplate wells and prevent local overheating during irradiation, a Peltier-cooled surface was used (Torrey Pines Scientific SC25 with microplate holder attachment). A full-spectrum solar light (1.5 AM) 16S-300-002 with XPS400 precision power supply from SolarLight Company, PA was used to provide the irradiation of test plates. Irradiation intensity and irradiation doses were measured with PMA2100 Radiometer and PMA2101 Detector (all from SolarLight Company, PA). Gapless backgrounds for the test microplates were assembled from white, light beige or black bands from Leneta 25 C skin tone cards. Cover made of about 2 mm thick opaque black polystyrene plastic with precision-drilled openings and inter-well fixator pegs was used to limit irradiation to test wells on the plate.

Choice of the fluorogenic probe determines the specificity of the test. Two probes have been used during testing to determine efficacy against different modes of sun light induced skin ageing and damage.

2′,7′-dichlorofluorescin diacetate (DCFDA) is a probe sensitive to a variety of peroxyl, peroxide, peroxynitrite and more complex peroxy products of oxidative damage to various biomolecules such as cell membrane phospholipids.

Singlet Oxygen Sensor Green Reagent (SOSGR) is a molecular probe with high specificity to singlet oxygen damage.

The choice of these probes is also convenient because when they are converted to fluorescent form, their excitation wavelengths are similar to each other, and emission wavelengths are also similar to each other, thus enabling the use of the identical microtiter plate reader protocol for plates prepared with either probe. For determination of fluorescence, a BioTek Synergy 2 microplate reader was used, with protocol using the 485/20 excitation filter and 528/20 emission filter.

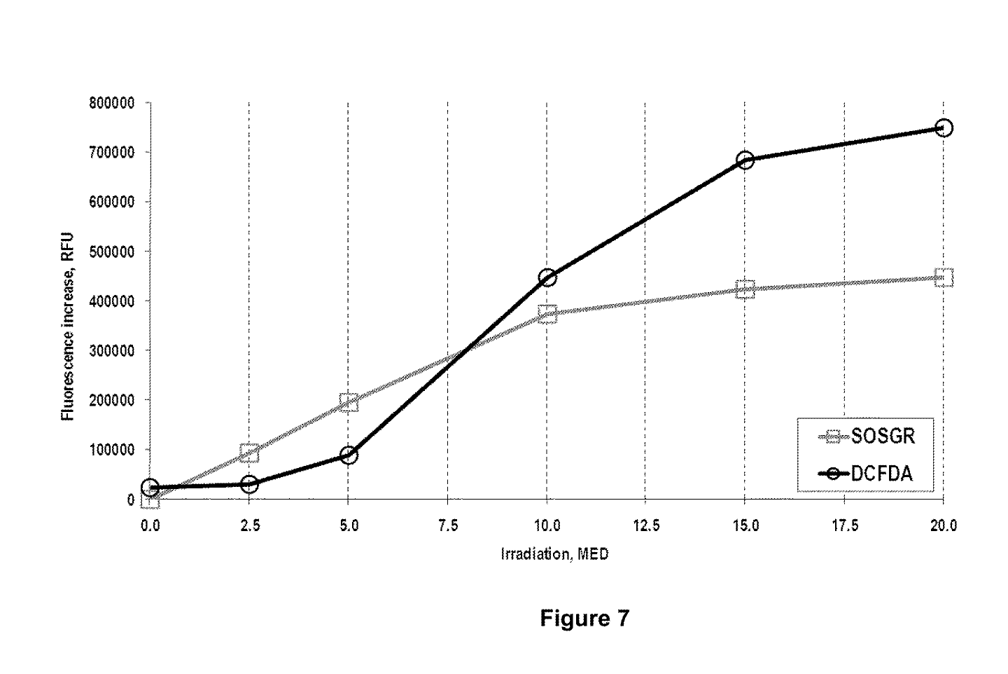

Different durations of irradiations at same intensity were tested to determine system response and find a suitable dose for continued testing, as illustrated in FIG. 7.

The exposure corresponding to 10 MEDs as calculated for the output of solar light simulator 16S-300-002 Solar Simulator (SolarLight Company, PA) that produces full spectrum (UV-VIS) sunlight (Air Mass, AM 1.5) was sufficient to generate readily detectable levels of fluorescence with either of molecular probes used in the study, with potential to detect UV-absorbing and anti-oxidant effects which would decrease the fluorescence and pro-oxidant effects which would increase it.

Therefore, all further testing was conducted using 10 MEDs as standard irradiation dose. Other irradiation doses can be successfully used as well.

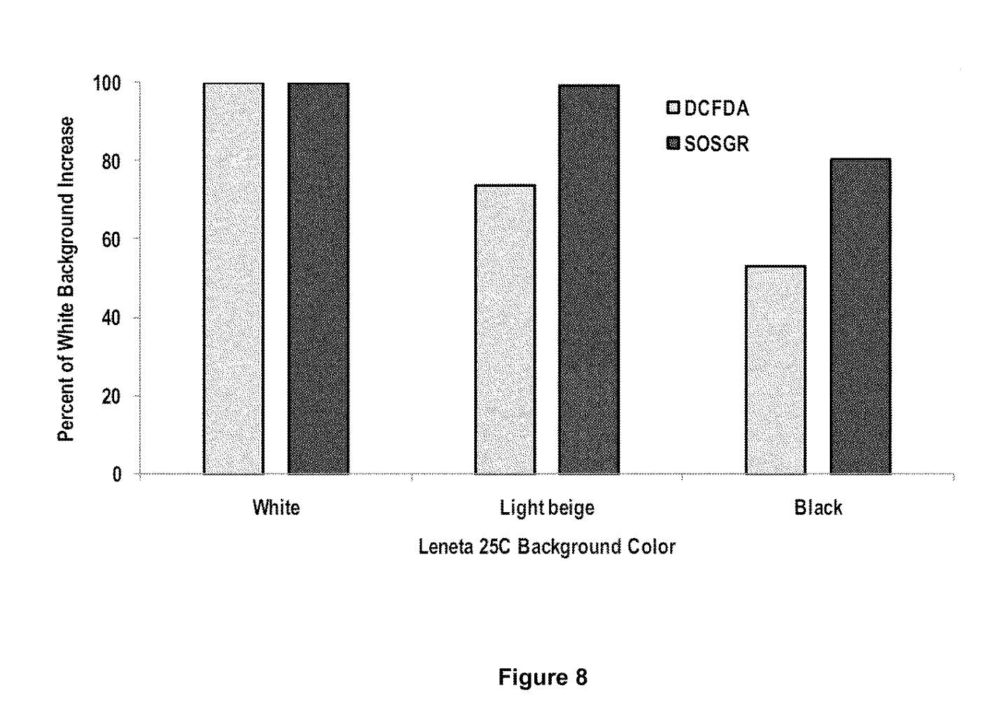

In addition to being responsive to changes in irradiation dose, these methods are responsive to color of the background used for the wells. Systems with both probes and vehicle control were tested on different backgrounds, as illustrated in FIG. 8.

This shows that choice of color background has a noticeable effect on the increase in probe fluorescence, which corresponds to probes being affected by sunlight induced free radical activity.

Dark (black) background color shows less increase in fluorescence, which corresponds to less free radical production, which include but are not limited to: peroxyl, peroxide, peroxynitrite and more complex peroxy products of oxidative damage to biomolecules such as cell membrane phospholipids; and singlet oxygen.

This trend is similar to one shown in Table III for photostability of sunscreens on the respective backgrounds.

For further testing of test articles, a light beige background was chosen. The details of the method were as follows.

The 2% w/w liposome solution was produced fresh for each test by sonicating (Sonics VibraCell VC750 20 KHz power supply, CV334 converter, 630-0220 probe) asolectin from soybeans (Sigma BioChemika 11145) in phosphate buffered saline (Invitrogen Gibco 10010) in temperature-stabilized bath on temperature-controlled magnetic stirrer (Torrey Pines Scientific HS40) at 600 RPM for 2 minutes at 100% amplitude.

For molar calculations, molecular weight of soybean asolectin from Sigma may be assumed to be similar to molecular weight of its principal component, phosphatidyl choline. These liposomes were used as cellular model for sun light induced lipid peroxidation because unsaturated lipids are present in the cellular membranes and extracellular matrix [Biplab Bose, Sanjiv Agarwal and S. N. Chatterjee. UV-A induced lipid peroxidation in liposomal membrane. Radiat Environ Biophys (1989) 28: 59-65, which is hereby incorporated by reference herein in its entirety].

Dichlorofluorescein diacetate (Sigma 35845) 0.1% w/v stock solution in anhydrous ethanol was made fresh daily and kept in the dark at 4 deg. C. The working solution of 0.0025% w/w DCFDA in PBS was prepared by diluting the stock solution in phosphate buffered saline (Invitrogen Gibco 10010) immediately before each test.

SOSGR (Invitrogen Gibco S36002) working solution was prepared before each test by adding 100 microliters methanol to a 100 microgram vial of SOSGR and diluting the resulting solution in 3124 microliters of phosphate buffered saline (Invitrogen Gibco 10010). SOSGR probe molar concentration in resulting working solution is equal to DCFDA molar concentration in 0.0025% DCFDA working solution as described above.

Test article dilutions were prepared in deionized water. Pure deionized water was used as vehicle control. Other solvents can be used they are compatible with the system.

Plate layout for each tested plate always included wells with vehicle control as well as wells for at least one test article. Half the wells for every tested substance including vehicle control were designated as “dark” wells that would not be irradiated. The other half were designated as “light” wells that would be subjected to irradiation. A stopwatch was used to ensure consistent timing in plate preparation and following steps. The plates were prepared by dispensing 10 microliters of test article dilution or vehicle control into wells, followed by 20 microliters of liposome solution and 45 microliters of working solution of a single probe.

Fluorescence readings at excitation wavelength were taken immediately for entire plate, recorded as fluorescence level before irradiation. The wells designated as “light” were irradiated using the solar light simulator, with cover placed to prevent irradiation of “dark” wells.

After the irradiation was complete, fluorescence readings of entire plate were taken again. Initial fluorescence readings were subtracted from these to calculate increase in fluorescence.

The increase in fluorescence for “dark” wells for a tested substance (including vehicle control) were averaged and subtracted from increase in fluorescence for “light” wells for same tested substance (including vehicle control) to calculate fluorescence increase due to irradiation.

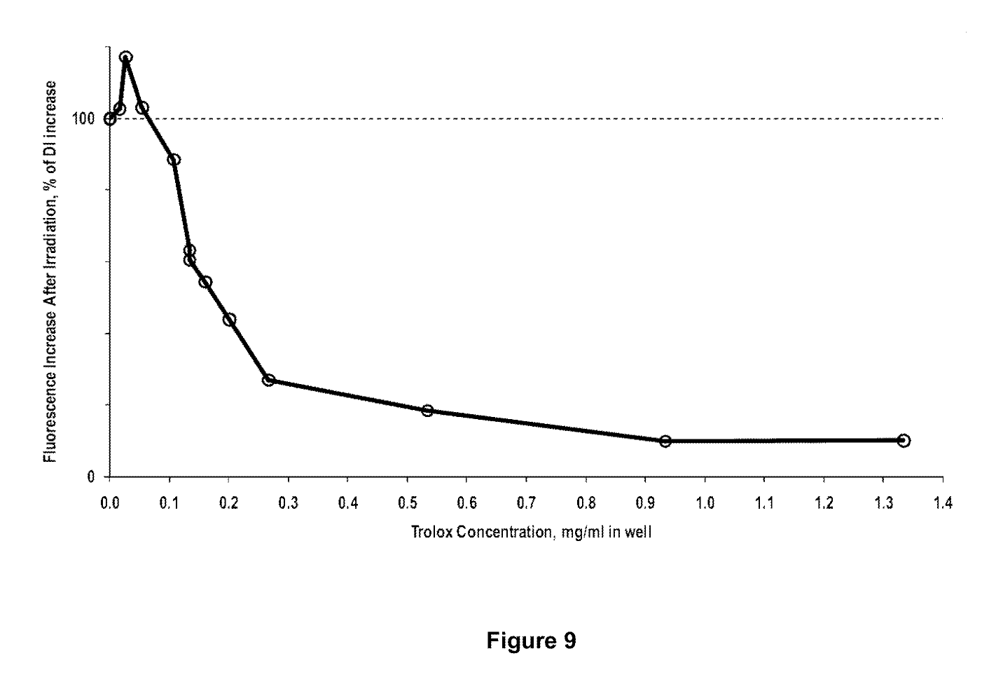

Comparing these figures allows one to determine whether a test article in a given concentration is compatible with the assay—for example, an incompatible substance may cause significant fluorescence increase in “dark” wells compared to vehicle control. Irradiation-induced fluorescence increases higher than those of vehicle control may point to pro-oxidant activities, and lower may point to anti-oxidant and UV-protective activities. Additionally, a material that shows apparent antioxidant or UV-protective activity in one concentration may act as apparent pro-oxidant or UV sensitizer in another concentration, as illustrated in FIG. 9.

One of the plausible mechanisms for this may involve molecules of antioxidant or UV-protecting substance being damaged in course of performing their intended functions resulting in production not of inert, but of reactive species. In low concentrations the net effect would contribute to further damage, while in higher concentrations net effect would be predominately determined by remaining intact molecules of the substance.

Performing the test with different dilutions of test article allows plotting the results of relative fluorescence increase versus concentration to more comprehensively determine the behavior of a test article in regards to UV-VIS-induced damage in in vitro model approximating some characteristics of skin and stratum corneum such as color skin type and diffuse reflectance and aspects of chemical composition.

Further modifications of this method may include but are not limited to: choice of fluorogenic, chromogenic, or otherwise indicative probes with different ROS specificity; choice of substrate approximating skin surface properties such as Vitro Skin® (N-19) suffused with a probe-containing solution; or utilization of this approach in various cell culture and skin equivalent systems.

### Example 5

### Activities of the Bioactive Ingredient Obtained from Fresh Camellia sinensis Against ROS

Bioactive ingredient Recentia™ Camellia sinensis Serum Fraction (CAS #1196791-49-7 with CAS definition: extractives and their physically modified derivatives that are protein-free, obtained by fractionation of the cell juice from Camellia sinensis) was obtained from fresh Camellia sinensis according to the process described in [Koganov, M., U.S. Pat. No. 7,473,435, Bioactive compositions form Theacea plants and processes for their production and use, which is hereby incorporated by reference herein in its entirety]. Recentia™ Camellia sinensis Serum Fraction was tested according to the Example 4 of present invention.

Two probes have been used to determine efficacy of this ingredient against different modes of sun light induced skin ageing and damage: DCFDA that is sensitive to peroxyl, peroxide, peroxynitrite and complex peroxy products of oxidative damage to various biomolecules such as cell membrane phospholipids and SOSGR with high specificity to singlet oxygen.

Light beige background was chosen to approximate color and diffuse reflectance characteristic of very light skin color type that is most susceptible to sun light induced skin ageing and photo damage.

Test results presented at Table V indicate that bioactive ingredient obtained from fresh Camellia sinensis demonstrated high efficacy against sun light induced ROS that include but are not limited to singlet oxygen and peroxy products of oxidative damage to various biomolecules such as membrane phospholipids; which was accompanied by absence of pro-oxidant activity.

TABLE V
Activities of the bioactive ingredient obtained from fresh
Camellia sinensis against sun light induced ROS

| | Dilution of test article with De- | | |
|---|---|---|---|
| | ionized Water volume/volume | Fluorescence VS. | |
| | (Concentration of test article, % | Control, % | |
| Test Aricle | weight/volume in well) | DCFDA | SOSGR |
| Control (de- | None | 100% | 100% |
| ionized water) | | | |
| Recentia ™ | 1/10 (1%) | 20-24% | 43-52% |
| Camellia | 1/30 (0.35%) | 50-53% | 55-61% |
| sinensis Serum | 1/90 (0.12%) | 67-70% | 72-79% |
| Fraction | | | |

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. An in vitro method for determining efficacy of a topical composition on a particular skin color type, said method comprising:
(a) providing an artificial skin apparatus configured to approximate color and diffuse reflectance characteristics of a predetermined human skin color type, said artificial skin apparatus comprising an artificial skin substrate combined with a color background that correlates to the human skin color type;
(b) applying a topical composition of interest to the artificial skin substrate of the artificial skin apparatus;
(c) pre-irradiating the topical composition applied to the artificial skin substrate; and
(d) analyzing the pre-irradiated topical composition for at least one efficacy parameter.

2. The method according to claim 1, wherein the artificial skin substrate is configured to have properties selected from the group consisting of surface properties that replicate those of human skin, surface topography (roughness) corresponding to that of human skin, contours that approximate human skin, roughened surface, and adapted for testing of the ultraviolet light absorbing and efficacy testing of topical compositions.

3. The method according to claim 1, wherein the artificial skin substrate is selected from the group consisting of VITRO SKIN® (N-19), a PMMA-based substrate, a quartz-based substrate, a polymer film having a thickness of about 100-1,000 μm and exhibiting at least 10% transmission of light having a wavelength of about 280-450 nm, a polypropylene-based substrate, and the like.

4. The method according to claim 1, wherein the artificial skin substrate is in the form of a single well or a plurality of wells.

5. The method according to claim 1, wherein the artificial skin substrate is in the form of a single well or a plurality of wells in combination with a layer of phospholipid liposomes and essential constituents of biological membranes or the like.

6. The method according to claim 1, wherein the artificial skin substrate is in the form of a single well or a plurality of wells in combination with a cell culture or a skin equivalent.

7. The method according to claim 1, wherein the color background correlates to a human skin color type selected from the group consisting of very light, light, intermediate, tan, brown, and black human skin color types.

8. The method according to claim 1, wherein the color background corresponds to a color of a Leneta Chart 25 C selected from the group consisting of white, light beige, dark beige, yellow beige, light brown, dark brown, and black.

9. The method according to claim 1, wherein said at least one efficacy parameter is effective to measure an activity selected from the group consisting of anti-ageing activity, photoprotective activity, sun protective activity, UVA/UVB protective activity, UVAI protective activity, photo stabilizing activity, and photosensitizing activity.

10. The method according to claim 1, wherein said at least one efficacy parameter is determined by using an assay selected from the group consisting of a diffuse transmittance assay, a diffuse reflectance in UV/VIS range assay, a fluorescence in UV/VIS range assay, a free radical assay, an antioxidant assay, a reactive oxygen species (ROS) assay, a cell culture-based assay, a skin equivalent-based assay, and the like.

11. The method according to claim 1, wherein said at least one efficacy parameter is measured by determining products of irradiation using spectrophotometric, chromatographic, mass spectroscopy, nuclear magnetic resonance, and/or electron paramagnetic resonance techniques.

12. The method according to claim 1, wherein the pre-irradiation of step (c) is conducted under natural, simulated sunlight, or artificial irradiation conditions.

13. The method according to claim 1, wherein the topical composition is selected from the group consisting of a single ingredient, a mixture of ingredients, and a formulation.

14. The method according to claim 1 further comprising:
(e) performing steps (a) through (d) for the same topical composition at least one additional time using a different color background, thereby yielding efficacy parameters of the topical composition on a plurality of different color backgrounds; and
(f) comparing the efficacy parameters obtained from step (e).

15. An artificial skin apparatus for determining efficacy of a topical composition on a particular skin color type, said apparatus comprising:
an artificial skin substrate; and
a color background that correlates to a human skin color type,
wherein said artificial skin substrate and said color background are combined to yield an artificial skin apparatus that approximates the color and diffuse reflectance characteristics of a predetermined human skin color type.

16. The apparatus according to claim 15, wherein the artificial skin substrate is configured to have properties selected from the group consisting of surface properties that replicate those of human skin, surface topography (roughness) corresponding to that of human skin, contours that approximate human skin, roughened surface, and adapted for testing of the ultraviolet light absorbing and efficacy testing of topical compositions.

17. The apparatus according to claim 15, wherein the artificial skin substrate is selected from the group consisting of VITRO SKIN® (N-19), a PMMA-based substrate, a quartz-based substrate, a polymer film having a thickness of 100-1,000 and exhibiting at least 10% transmission of light having a wavelength of 280-450 nm, a polypropylene-based substrate, and the like.

18. The apparatus according to claim 15, wherein the artificial skin substrate is in the form of a single well or a plurality of wells.

19. The apparatus according to claim 15, wherein the artificial skin substrate is in the form of a single well or a plurality of wells in combination with a layer of phospholipid liposomes and essential constituents of biological membranes or the like.

20. The apparatus according to claim 15, wherein the artificial skin substrate is in the form of a single well or a plurality of wells in combination with a cell culture or a skin equivalent.

21. The apparatus according to claim 15, wherein the color background correlates to a human skin color type selected from the group consisting of very light, light, intermediate, tan, brown, and black human skin color types.

22. The apparatus according to claim 15, wherein the color background corresponds to a color of a Leneta Chart 25 C selected from the group consisting of white, light beige, dark beige, yellow beige, light brown, dark brown, and black.

23. A kit for determining efficacy of a topical composition on a particular skin color type, said kit comprising:
an artificial skin apparatus according to claim 15; and
instructions for using the artificial skin apparatus to determine efficacy of a topical composition of interest on one or more different human skin color type.
